(19) United States
(12) Patent Application Publication
(10) Pub. No.: US 2013/0317405 A1
(43) Pub. Date: Nov. 28, 2013

(54) MODULAR WOUND DRESSING

(71) Applicants: Phong V. Ha, Hudson, WI (US); Donald G. Peterson, Shoreview, MN (US)

(72) Inventors: Phong V. Ha, Hudson, WI (US); Donald G. Peterson, Shoreview, MN (US)

(21) Appl. No.: 13/981,041

(22) PCT Filed: Feb. 10, 2012
(86) PCT No.: PCT/US12/24660

§ 371 Date: Jul. 22, 2013

Related U.S. Application Data
(60) Provisional application No. 61/443,288, filed on Feb. 16, 2011.

(51) Int. Cl. A61F 13/00

(52) U.S. Cl. CPC A61F 13/00
USPC 602/42

(57) ABSTRACT

The disclosed medical dressing has at least two thin film backings in overlapping relation to one another. The backings entirely surround an opening to form a window. In one embodiment, the first and second backings are a conformable film each formed in a generally C-shape such that the open ends of the C-shape are laterally aligned with one another and form an opening at least partially surrounded by the first and second backing. In use, the opening generally is aligned with a wound. The medical dressing, being constructed from at least two backings, allows for control of the desired opening side during application of the backings by overlapping portions of the backings.

### FIELD

The present disclosure relates to a medical dressing comprising two backing layers. In particular, the two backing layers are in overlapping relation to one another and entirely surround an opening to form a window.

### BACKGROUND

Transparent film dressings are widely used as protective layers over wounds because they facilitate healing in a moist environment while acting as a barrier to contaminating liquids and bacteria. The films are also used as surgical drapes because of their barrier properties. Dressings and drapes fitting the above description are available under a number of trade names such as TEGADERM™ (3M Company, St. Paul, Minn.), BIOCLUSIVE™ (Johnson & Johnson Company, New Brunswick, N.J.), and OP-SITE™ (T. J. Smith & Nephew, Hull, England).

The polymeric films used in those dressings and drapes are conformable, i.e., the films are extremely thin, flexible and supple. They are typically supplied with a releasable protective liner covering the adhesive coated surface of the film. When the liner is removed, the adhesive coated film tends to wrinkle and adhere to itself, interfering with the smooth, aseptic application of the dressing to a patient's skin. Various delivery systems have been proposed to address this problem such as those disclosed in U.S. Pat. No. 6,685,682. The use of a removable carrier, which does not require tearing of the film after it has been placed on the patient, avoids the problems described above. The carrier also aids in accurate placement of the dressing on a patient. Even with the carrier however, the dressings can be difficult to place on an irregular body surface, such as a joint (e.g., knee, elbow) or shoulder.

In addition, the length of time over which the medical dressings may remain in place over wounds may be limited by many factors. Among the factors that may limit the usable life of a medical dressing is the accumulation of fluids within the wound. Some medical dressings include absorbent components to absorb wound fluid. Some medical dressings have included the use of negative pressure wound therapy in which fluids are removed from beneath the wound dressings without requiring removal of the dressings from the patient. Dressings adapted for delivery of negative pressure wound therapy (such as those described in, e.g., U.S. Pat. Nos. 4,969,880; 5,261,893; 5,527,293; and 6,071,267 (all to Zamierowski)) often have constructions that can compromise the sterility of the wound over which they are placed. These products often require a tube or wound drain that is introduced either through a multi-piece dressing or under a single piece dressing. In either case, it is difficult to obtain a good seal between the tube or wound drain and, during treatment, air can leak into the wound. That air can carry contamination into the wound and/or impair the effectiveness of the pressure-based therapy. These effects can be compounded by wounds on or around irregular surfaces, such as the knee, elbow, shoulders, heel, and ankle.

Certainly, the size and shape of the wounds can vary. Therefore, clinics and hospitals must maintain a number of different sized dressings for various patient needs. Having many different sized dressings adds inventory and therefore cost to hospital.

A need remains for a medical dressing that can be more effectively supported and/or more conformable for application to irregular surfaces, and particularly those associated with a joint, such as a knee, ankle or elbow, and particularly in wound therapy applications that can accommodate various sized wounds.

### SUMMARY

The disclosed medical dressing comprises at least two thin film backings in overlapping relation to one another. The two thin film backings entirely surround an opening to form a window that surrounds a wound. In one embodiment, the first and second backings are a conformable film each formed in a generally C-shape such that the open ends of the C-shape are laterally aligned with one another and form an opening at least partially surrounded by the first and second backing. In use, the opening generally is aligned with a wound. The medical dressing, being constructed from at least two backings, allows for control of the desired opening size during application of the backings by overlapping portions of the backings. In one embodiment, the medical dressing further includes a third thin film backing that is in overlapping relation with the first or second backing.

In one embodiment, a pouch is provided that is secured to a second major surface of the first backing and a second major surface of the second backing and completely surrounds the opening. The pouch forms a cavity at the opening. In one embodiment the pouch is a self-supporting substrate. In one embodiment the pouch is laterally expandable or collapsible.

In one embodiment, a filling element is included for placement within the pouch. The filling element may be secured within the pouch. The filling element may be laterally expandable or collapsible. The filling element may be an absorbent material, a barrier element, or a wound packing material.

In one embodiment, the pouch is a separately applied over the opening. In such an embodiment, the self-supporting substrate can be a single unit that can expand or contract to fit various opening sizes or the self-supporting substrate can comprise at least two portions that themselves can overlap one another.

In one embodiment, a portion of the pouch is incorporated with the first backing and a portion of the self-supporting substrate is incorporated with the second backing such that both the backings and portions of pouch are in overlapping relation to one another and the backings form an opening that the pouch covers.

In one embodiment, the medical dressings and medical dressing kits described herein can also be used to provide a ported medical dressing for placement over a wound or other body site where controlled fluid access is desired. That fluid access is preferably available without removing or otherwise disturbing the medical dressing. In particular, the controlled fluid access provided by medical dressings described herein may be useful to remove fluids from the wound (as in, e.g., negative or reduced pressure therapies), to provide one or more gases (e.g., oxygen, nitric oxide, ozone, etc.) to a wound site, to provide one or more liquids (e.g., saline, etc.), and/or to provide one or more active agents (e.g., carried in a liquid or gas) to a wound site. The pouch may comprise a valve to aid in fluid flow.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments.

As used herein, “a,” “an,” “the,” “at least one,” and “one or more” are used interchangeably. The term “and/or” (if used) means one or all of the identified elements/features or a combination of any two or more of the identified elements/features.

### BRIEF DESCRIPTION OF THE DRAWINGS

FIG. 1 is a top plan view of an embodiment of a medical dressing comprising a first backing and a second backing;

FIG. 2 is a schematic cross section of the medical dressing of FIG. 1;

FIG. 3 is an exploded perspective view of a medical dressing;

FIG. 4 is a top perspective view of one embodiment of a pouch;

FIG. 5 is a top view of an expandable filling element laterally unstretched;

FIG. 6 is a perspective view of an expandable filling element laterally stretched;

FIG. 7 is a top plan view of another embodiment of a medical dressing comprising a first backing and a second backing each comprising a portion of a pouch;

FIG. 8 is a top view of an embodiment of a medical dressing comprising a first backing, second backing, and third backing surrounded an opening containing a wound.

While the above-identified drawings and figures set forth embodiments of the invention, other embodiments are also contemplated, as noted in the discussion. In all cases, this disclosure presents the invention by way of representation and not limitation. It should be understood that numerous other modifications and embodiments can be devised by those skilled in the art, which fall within the scope and spirit of this invention.

The figures may not be drawn to scale.

### DETAILED DESCRIPTION

The disclosed medical dressing comprises at least a first thin film backing and a second thin film backing. In use, the first backing and second backing are in overlapping relation to one another and entirely surround an opening. The backings surround the wound and the opening contains the wound. Opening means that the surface containing the wound does not have a directly overly film, such that the backings form a perimeter or window around the opening and therefore surrounds the wound.

In one embodiment, the first and second backings are a conformable film each formed in a generally C-shape. C-shape means a film structure having outwardly extending legs such that the backing has an open end with the legs spaced from one another and a closed end where the legs are connected. It is understood that by “C-shape” the shape of the dressing could be in any general shape having an open end and a connected end, such as a C, U, V shape. In use, at least two backings are provided such that the open ends of the C-shape are laterally aligned with one another and surround the opening. Further, in use the backings form a perimeter or window around the opening to surround the wound.

The medical dressing, being constructed from at least two backings, allows for control of the desired opening size during application of the backings by overlapping portions of the backings. In one embodiment, the medical dressing further includes a third thin film backing that is in overlapping relation with the first or second backing.

In one embodiment, the medical dressing further comprises a pouch that is placed over the opening. The pouch may hold a filling element, which in one embodiment could be absorbent material, a barrier element, or a wound packing material. The pouch may be a self-supporting substrate.

The medical dressing and pouch are well suited to application over a convex surface of a patient, such as a patient's knee. The medical dressing permits a wound therapy, such as negative pressure therapy, to be located against the wound, while the pouch aids in forming a seal around the convex or irregularly shaped area surrounding the wound.

In some implementations the medical dressing comprises a first and second backing layer, an adhesive layer on each of the backing layers facing the wound, and a pouch. Once the backing layers are connected in overlapping relation to one another, the adhesive layer on the backing layers form a perimeter around an opening that the pouch covers. The perimeter formed by the adhesive layer and backing layers keeps the pouch properly positioned, and also helps maintain a sterile environment around the wound.

The adhesive layer and backing layer are typically extremely thin, and generally very flexible.

In one embodiment, the pouch is secured to the backing layers and remains on the medical dressing after the dressing is applied. In one embodiment, the pouch comprises a first portion secured to the first backing layer and a second portion secured to the second backing layer. In use, the pouch portions, like the backing layers are in overlapping relation to one another to create the sealed, sterile environment over the wound.

The medical dressing is highly conformable, and is easy and fast to apply to a wound site. The overlap between the first backing layer and second backing layer can be adjusted so that the opening, which contains the wound, can be adjusted for different sized wounds. If the pouch is separate from the backing layers, the pouch can be provided over the opening and therefore over the wound. In one embodiment, the pouch and filling element, if included, may be laterally adjustable to accommodate various opening sizes.

The medical dressings described herein can be used to provide a sealed environment over a wound or other body site. Fluid removed from the sealed environment may include gases and/or liquids (which may contain dispersed solid particles such as necrotic tissue, blood clots, etc.). The fluid removal can be performed through an opening in the dressing, such as a valve, without removing or otherwise disturbing the medical dressing.

As used herein, the term “sealed environment” means that fluids (and solids) from the ambient atmosphere surrounding the exterior of a medical dressing attached over a wound cannot freely enter the sealed environment. The sealed environment preferably includes a hermetic seal between the medical dressing and the surface surrounding the wound such that a negative pressure can be maintained in the sealed environment. It may, for example, be preferred that the medical dressing be capable of holding (at least temporarily as described herein) a vacuum of 100 mmHg (i.e., a pressure that is 100 mmHg below atmospheric pressure) and perhaps a vacuum as much as 200 mmHg. Although some conventional medical dressings can provide such a sealed environment, the medical dressings described herein can do so while also offering the opportunity to remove fluids (liquids and/or gases) from the sealed environment through an opening, such as a valve, provided as a part of the medical dressing.

As used herein, the term “self supporting” means that the substrate can hold a definable shape in the x-, y-, and z-plane in the absence of any applied force. In some embodiments, a self supporting substrate may withstand some level of applied pressure or force.

As used herein the term “vacuum” refers to pressures less than the surrounding atmospheric pressure. Preferably the pressure is reduced by 5-250 mm mercury (Hg) (e.g., down to an absolute pressure of 500-740 mmHg, but this will depend on the atmospheric pressure). When the pressure is reduced by more than 250 mmHg the patient may experience pain. Thus, preferably the pressure is not reduced by more than 200 mmHg and more preferably by not more than 175 mm Hg. Preferably, the pressure is reduced by at least 5 mmHg, 25 mmHg, more preferably at least 50 mmHg and most preferably at least 75 mm Hg in order to remove sufficient interstitial fluid.

Fluid removal from the sealed environment may be useful to provide negative or reduced pressure therapies to a wound over which the medical dressing is located. Fluid removed from the wound dressing may include gases and/or liquids (which may contain dispersed solid particles such as necrotic tissue, blood clots, etc.). The fluid removal can be performed without removing or otherwise disturbing the medical dressing.

With the medical dressing and pouch described herein, the sealed environment created by a medical dressing may be maintained at a negative pressure (i.e., pressure below the ambient atmospheric pressure) in the absence of active vacuum source in fluid communication with the sealed environment. In other words, the medical dressings may be used to maintain a sealed environment with a negative or reduced pressure in the periods between active removal of fluids from the sealed environment. As a result, the medical dressings can provide a negative or reduced pressure environment with only intermittent or periodic fluid removal.

Although the magnitude of the negative pressure maintained in the sealed environment by the medical dressings will typically deteriorate over time (after reaching a maximum during that active removal of fluids from the sealed environment), it may be preferred that the medical dressing be capable of maintaining the negative pressure for at least some significant period of time. In some embodiments, it may be preferred that the medical dressing be capable of maintaining at least some level of negative pressure in the sealed environment (in the absence of active fluid removal) for a period of 1 minute or more, 5 minutes or more, 10 minutes or more, 15 minutes or more, 30 minutes or more, or even 60 minutes or more.

Deterioration of the negative pressure within the sealed environment defined by the medical dressing may be caused by a variety of sources. For example, some of the deterioration may be due to the diffusion of gas into the sealed environment through the backing of the medical dressing and/or the adhesive attaching the medical dressing to a subject. Another source of negative pressure deterioration in the sealed environment may be caused by gases and/or liquids entering the sealed environment from the subject (i.e., through the wound itself and/or the tissue surrounding the wound).

Among the potential advantages that may be associated with use of the medical dressings of the invention is that, in some instances, the negative pressure may advantageously pull the edges of acute incisional wounds together, thus potentially providing faster healing, reduced infection rates, and/or improved cosmetic results (reduced scarring).

In one embodiment, to use the medical dressing, the first backing is applied to a wound-containing surface, a second backing is applied in overlapping relation to the first backing. The first backing and second backing entirely surround the wound and form an opening surrounded by the backings. In one embodiment, a pouch is applied over the opening and therefore over the wound. In one embodiment, a first portion of a pouch is provided on the first backing and a second portion of a pouch is provided on the second backing and the first portion of the pouch is in overlapping relation to the second portion of the pouch. After the medical dressing is applied over a wound, in one embodiment, fluid from the sealed environment is removed from the surface of the wound. In one embodiment, a valve is provided in the medical dressing to remove fluid from the surface of the wound. In some embodiments such as those provided herein, the method of removing fluid from the internal volume comprises removal of air such that the pressure within the sealed environment is below atmospheric pressure, and the method of removing fluid from the internal volume comprises removal of wound exudate from the wound.

Although the medical dressings may be used to provide negative pressure wound therapy, in some instances fluids or other materials may potentially be delivered into the sealed environment through the medical dressing. For example, delivery devices such as pipettes, needles, etc. may be used to pierce the backing of the medical dressing, with the fluids or other materials delivered into the sealed environment through the delivery devices. It may be preferred that the delivery of materials into the sealed environment through the medical dressing does not functionally compromise the ability of the medical dressing to define a sealed environment as described herein.

To provide resealable access to the sealed environment through the medical dressing, the material used for the medical dressing backings may, for example, be self-sealing such that an opening formed through the backing(s) seals upon removal of the delivery device (in the manner of, e.g., a septum). In other instances, a closure element may be applied over the external surface of the medical dressing backing(s) after the delivery device is removed to close any opening formed through the backing by the delivery device.

It is contemplated that fluids are delivered through the septum by inserting a tube similar to a vascular access catheter in which the catheter is inserted through the septum with the assistance of a blunt needle or introducer which is subsequently removed, leaving behind the blunt end relatively flexible catheter tubing. In this manner, no needle remains in place on the patient.

Fluids delivered to the sealed environment through the medical dressing may include gases (e.g., oxygen, nitric oxide, ozone, etc.) and/or liquids (e.g., saline, water, etc.). Particulates may, in some instances, also be delivered to the sealed environment if, e.g., they are entrained within a fluid delivered into the sealed environment.

### Illustrative Exemplary Embodiments

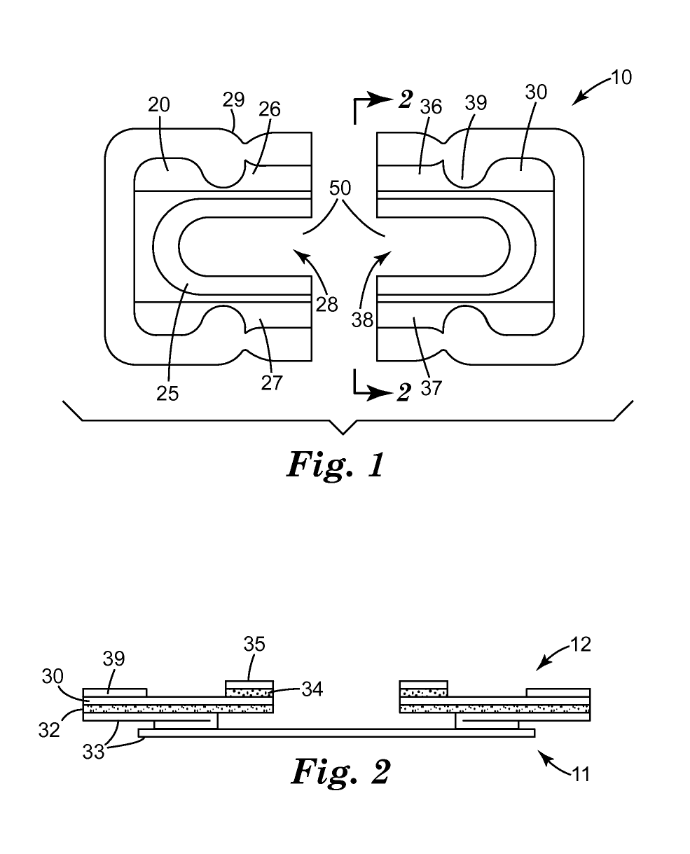

FIG. 1 is a top plan view of an embodiment of a medical dressing 10 comprising a first backing 20 and a second backing 30. FIG. 2 is a schematic cross section of the medical dressing 10 of FIG. 1 along line 2-2. The medical dressing 10, comprising both the first and second backings 20, 30 includes a first major surface 11 that faces the wound containing surface 13 and a second major surface 12 opposite the first major surface.

The first backing layer 20 comprises an adhesive layer 22 on all or a portion of the surface of the backing layer 20 that faces the wound 13 during use. The first backing layer 20 shown is in generally a C-shape having outwardly extending legs 26, 27 such that the first backing layer 20 has an open end 28 with the legs 26, 27 spaced from one another and a closed end where the legs 26, 27 are connected. In the area where the legs 26, 27 separate from one another the backing 20 is essentially free of the thin film and is therefore open.

The second backing layer 30 comprises an adhesive layer 32 on all or a portion of the surface of the backing layer 30 that faces the wound 13 during use. The second backing layer 30 shown is in generally a C-shape having outwardly extending legs 36, 37 such that the second backing layer 30 has an open end 38 with the legs 36, 37 spaced from one another and a closed end where the legs 36, 37 are connected. In the area where the legs 36, 37 separate from one another the backing 30 is essentially free of the thin film and is therefore open.

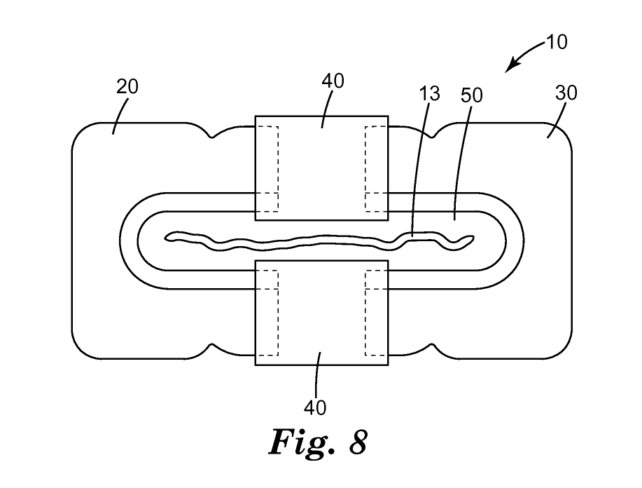

In use, the first backing layer 20 is applied to a wound containing surface 13 and the second backing layer 30 is applied in overlapping relation to the first backing layer 20 (similar to shown in FIG. 8). Generally, the first legs 26, 36 laterally align and in the embodiment depicted in FIG. 1 overlap and the second legs 27, 27 laterally align and in the embodiment depicted in FIG. 1 overlap. As will be appreciated, the amount of overlap can be adjusted to arrive at an opening 50 of various sizes to accommodate wounds 13 of various sizes.

In some embodiment, a third backing layer 40 can be included (see FIG. 8) that comprises an adhesive layer 42 on all or a portion of the surface of the backing layer 40 that faces the wound 13 during use. The third backing layer 40 shown is in generally linear extending legs 46, 47 such that the third backing layer 30 provides an ability to extend the separation of the first backing layer 20 from the second backing layer 30 while providing a complete perimeter around the opening 50. It is understood that the third backing layer 40 could comprises any number of shapes and sizes to allow for expansion of the size and shape of the opening 50. For example, the third backing layer 40 may include a T or Y extension to branch from laterally aligned first and second backings 20, 30. The third backing layer 40 may include adhesive on one or both of the first and second major surfaces. Alternatively, an adhesive is not necessary on the second major surface (as shown) and instead the pouch 60 would include the adhesive.

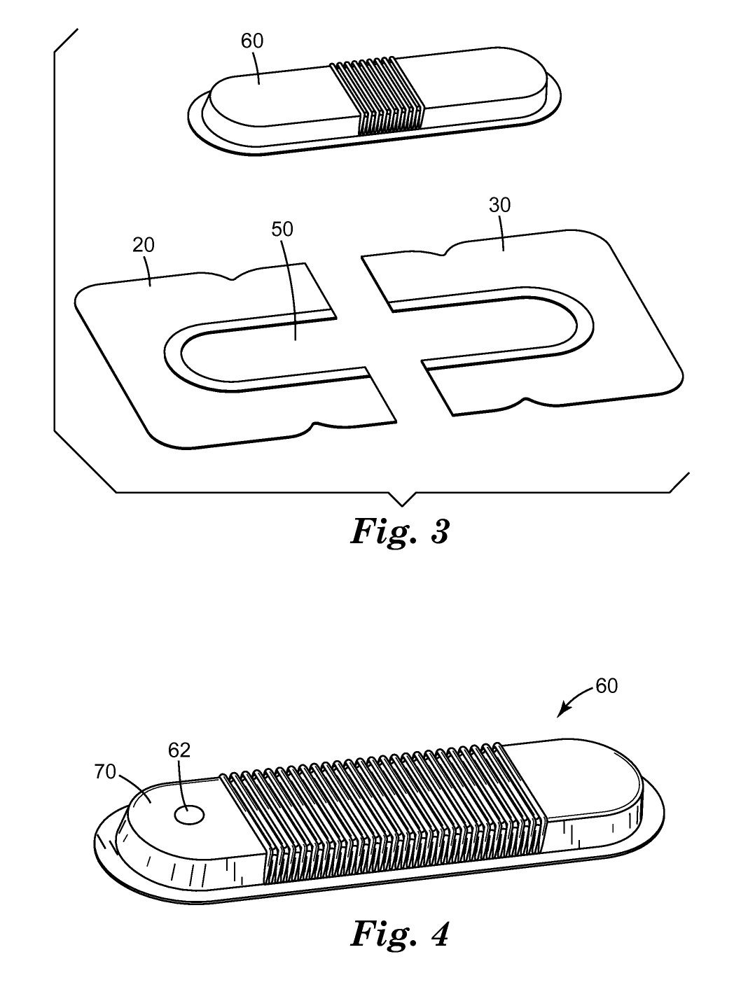

A pouch 60 may be used to cover the opening 50. The pouch 60 may be included either separately, as would be with FIGS. 1 and 3, or integrally, as would be with FIG. 7. In the embodiment shown in FIG. 1, an adhesive 24 is included on the second major surface 12 of the first backing 20 and an adhesive 34 is included on the second major surface 12 of the second backing 30 for connection with a pouch 50. The adhesive 24, 34 may be provided on the backing 20, 30 or maybe provided on the pouch 60.

For embodiments, such as shown in FIGS. 3 and 4 where the pouch 60 is a single unit separately applied over the opening 50, the pouch 60 may be laterally adjustable to accommodate the adjustable size of the opening 50 created by the first backing 20 and second backing 30. For example, such as shown in FIG. 4, the pouch 60 may include accordion-like peaks and valleys that allow for lateral extension of the pouch. In some embodiments, the accordion-like structure also provides flexibility of the pouch 60 once applied to a patient. It is understood that other mechanisms could be used to arrive at a laterally adjustable pouch 60, such as a stretchable or elastic material used for contraction of the pouch 60.

For embodiments, such as shown in FIG. 6, the pouch 60 includes a first portion 64 connected with the first backing 20 and a second portion 66 connected with the second backing 30. If a third backing 40 or additional backings are included then pouch portions could be provided on those backings as well. At one or both of the first or second portions 64, 66 of the pouch 60 an adhesive is included on the first major surface. Therefore, when the first and second backing are applied in overlapping relation to one another, the pouch portions 64, 66 are applied in overlapping relation to one another. The seal provided by the various adhesives and overlapping nature results in a sealed environment surrounding the wound 13.

FIG. 3 is an exploded perspective view of the medical dressing of FIGS. 1 and 2 with an overlying pouch 60 for placement over the opening 50. It is understood, that first the first backing 20 and second backing 30 would be placed in overlapping relation to one another over a wound containing surface 13. Then the pouch 60 could be applied over the opening 50.

FIG. 4 is a top perspective view of one embodiment of a pouch 50. The pouch may be a thin film material similar to that of the backings 20, 30 or the pouch may be a self-supporting substrate. Extensive disclosure of a self supporting substrate suitable for use in this application is disclosed in WO2010/147930, the disclosure of which is herein incorporate by reference.

The dressing 10 may include a valve 62, which is an opening to the pouch 50 as shown in FIG. 4. The valve 62 (preferably be a one-way valve) may be used to provide negative pressure therapy to a wound over which the dressing 10 is placed as described herein. The medical dressing 10 may include more than one valve 62. It may be preferred that the valve 62 be capable of being used one, two or more times to remove fluids from the pouch 60 without requiring that the medical dressing 10 be removed and without requiring the constant removal of fluid to maintain a negative pressure within the sealed environment. For example, fluid can be removed from the pouch 60 through the valve 62, with the valve being allowed to close when the fluid removal terminates. As additional fluid accumulates in the sealed environment, it can be removed through the valve 62. One exemplary embodiment of a valve 62 that may be used in a medical dressing 10 is disclosed WO 2009/124125, incorporated by reference in its entirety.

Although the magnitude of the negative pressure maintained in the sealed environment by the dressing 10 will typically deteriorate over time (after reaching a maximum during that active removal of fluids from the sealed environment through the valve 62), it may be preferred that the dressing 10 be capable of maintaining the negative pressure for at least some significant period of time. In some embodiments, it may be preferred that the dressing 10 be capable of maintaining at least some level of negative pressure in the sealed environment (in the absence of active vacuum source) for a period of 1 minute or more, 5 minutes or more, 10 minutes or more, 15 minutes or more, 30 minutes or more, or even 60 minutes or more.

It may be preferred that the fluid removal place the sealed environment at a negative pressure as discussed herein, although such a condition is not necessarily required. For example, the fluid removal may be limited to removing fluids such as wound exudate, blood, etc. from the sealed environment without necessarily resulting in a negative pressure condition within the sealed environment.

A filling element 70 may be included within the pouch 60, such as shown in FIG. 4. The filling element 70 may be an absorbent material, barrier element, or wound packing material (all described below). To accommodate various sizes of a pouch 60, due to the ability to create various sized openings 50, in one embodiment, the filling element 70 is laterally expandable or compressible.

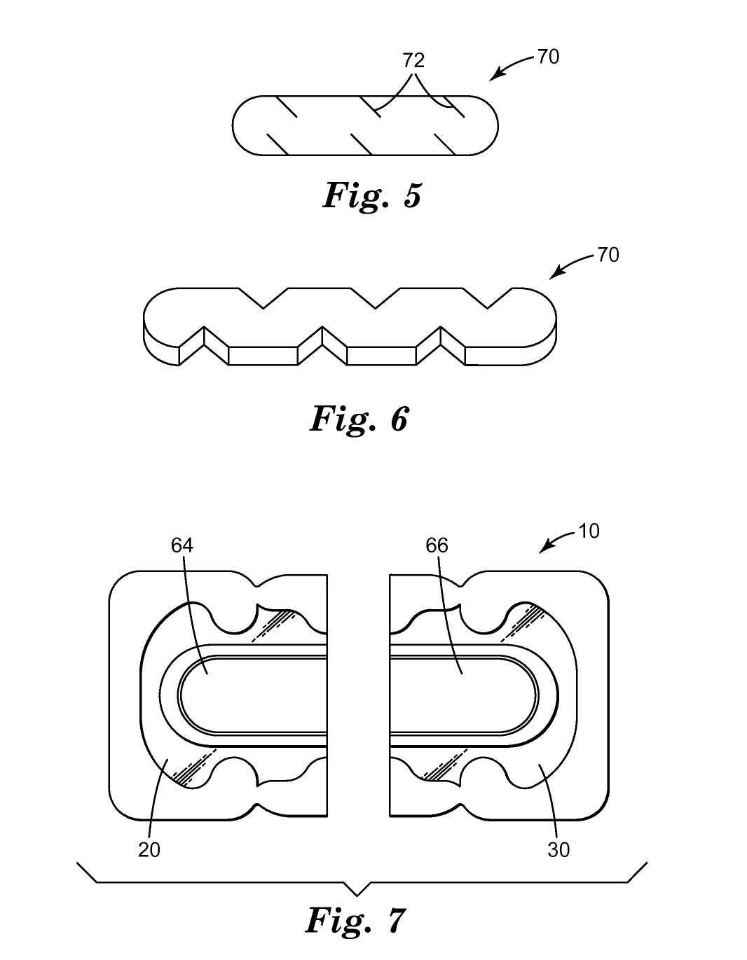

FIG. 5 shows a filling element 70 with inwardly extending cuts 72. FIG. 6 shows the filling element 70 of FIG. 5 laterally extended. The cuts 72 allow for a lateral extension of the filling element 70. The laterally extended filling element 70 could be placed with in a pouch 60 and secured within a pouch 60 if necessary to maintain the lateral extension. It is understood that a number of other mechanisms could be used to provide a filling element capable of lateral extension, such as an elastic material.

The filling element 70 may be a resiliently compressible material that, e.g., compresses or shrinks as a vacuum (negative pressure) is provided within the pouch and that attempts to return to at least a portion of its pre-compression size because of its resilient nature. For example, the filling element 70 may be a resilient foam (open or closed cell, although preferably open cell), nonwoven material, spring, or other structure that can be compressed, but that also is resilient such that it will attempt to return to at least a portion of its pre-compressed size (e.g., the resilient material has a spring constant).

In some embodiments, it may be preferred that the medical dressing include absorbent material to absorb fluids (e.g., liquids) entering the sealed environment. Examples of potentially suitable absorbent materials may include, but are not limited to, hydrophilic foams, woven materials, nonwoven materials, etc. and combinations thereof. It may be preferred that the absorbent material be both absorbent and capable of releasing at least some (preferably a majority) of any absorbed fluids when a vacuum is applied to the sealed environment through a valve. By releasing absorbed fluids during the removal of fluids from the sealed environment, the ability of the absorbent material to absorb fluids may be regenerated—which may prolong the useful life of the medical dressing.

The medical dressing 10 typically includes release liners 23, 25, 33, 35. The release liners 23, 25, 33, 35 covers the adhesive containing surface of the medical dressing. The release liners 23, 25, 33, 35 may be a single piece or multiple piece release liner, and may be part of or laminated to the package (not shown) containing the dressing 10, or merely enclosed along with the dressing within the package. The release liners 23, 25, 33, 35 keeps the adhesive 22, 24, 32, 34 clean during storage and shipping of the medical dressing 10. Once the release liners 23, 25, 33, 35 and dressing 10 are separated, only a carrier 29, 39 (as described further below) provide significant rigidity to the backing layers 20, 30.

The first backing layer 20 and second backing layer 30 are typically a thin film. The first backing layer 20 can include a carrier 29 and the second backing layer 30 can include a carrier 39 that provide structural support to the backing layers 20, 30.

The medical dressing 10 is typically applied to a patient by first cleaning the wound and making sure the area around the wound is ready to receive a dressing. The release liner 23 is then removed from the first backing 20 exposing the adhesive 22 that is secured to the skin surrounding a wound 13. The carrier 29, if included is removed. The release liner 33 is removed from the second backing 30 exposing the adhesive 32 that is secured to the skin surrounding a wound 13. The second backing 30 is in overlapping relation to the first backing 20 such that a perimeter formed of the first and second backings surrounds the opening 50. The pouch 60, which may contain the filling element 70 is applied over the opening 50. If needed the pouch and optional filling element 70 may be laterally stretched or contracted to fit in the pouch 60.

Suitable method of making various components of a backing layer and pouch are disclosed in WO 2010/147930, the disclosure of which is herein incorporated by reference.

### Backing Materials

The wound dressings are useful in connection with any conformable backing that provides a sufficiently impermeable barrier to the passage of liquids and at least some gases. Representative backings may include non-woven and woven fibrous webs, knits, films, foams polymeric films and other familiar backing materials. The preferred backing materials include thin elastomeric backings. These types of backings help ensure conformability and high adhesion around the wound site. Preferred backing materials may be translucent or transparent polymeric films including polyurethanes (e.g. ESTANE), polyether polyesters (e.g. HHTREL), polyether amides (e.g. PEGAX) as well as polyolefins (e.g. ENGAGE). The backing can be a high moisture vapor permeable film backing. U.S. Pat. No. 3,645,835 describes methods of making such films and methods for testing their permeability.

The backing advantageously should transmit moisture vapor at a rate equal to or greater than human skin. In some embodiments, the adhesive coated backing layer transmits moisture vapor at a rate of at least 300 g/m²/24 hrs/37° C./100-10% RH, frequently at least 700 g/m²/24 hrs/37° C./100-10% RH, and most typically at least 2000 g/m²/24 hrs/37° C./100-10% RH using the inverted cup method.

The backings used may be high moisture vapor permeable film backings. Issued U.S. Pat. Nos. 3,645,835 and 3,645,835 describe methods of making such films and methods for testing their permeability. The film (and any adhesive used thereon as described herein) may transmit moisture vapor at a rate equal to or greater than human skin. The adhesive-coated film may, e.g., transmit moisture vapor at a rate of at least 300 g/m²/24 hrs/37° C./100-10% RH, more preferably at least 700 g/m²/24 hrs/37° C./100-10% RH, and most preferably at least 2000 g/m²/24 hrs/37° C./100-10% RH using the inverted cup method as described in U.S. Pat. No. 4,595,001.

The backing layer is generally conformable to anatomical surfaces. As such, when the backing layer is applied to an anatomical surface, it conforms to the surface even when the surface is moved. The backing layer is also conformable to animal anatomical joints. When the joint is flexed and then returned to its unflexed position, the backing layer typically stretches to accommodate the flexion of the joint, but is resilient enough to continue to conform to the joint when the joint is returned to its unflexed condition.

A description of this characteristic of backing layers can be found in issued U.S. Pat. Nos. 5,088,483 and 5,160,315, the disclosures of which are hereby incorporated by reference. As discussed, particularly preferred backings are elastomeric polyurethane, polyester, or polyether block amide films. These films combine the desirable properties of resiliency, high moisture vapor permeability, and transparency found in preferred backings.

Commercially available examples of potentially suitable backing materials may include the thin polymeric film backings sold under the tradenames TEGADERM (3M Company), BIOSITE (Johnson & Johnson Company), OPSITE (Smith & Nephew), etc. Many other backings may also be used, including those commonly used in the manufacture of surgical incise drapes (e.g., incise drapes manufactured by 3M Company under the tradenames STERIDRAPE and IOBAN), etc.

Because fluids may be actively removed from the sealed environments defined by the medical dressings of the present invention, a relatively high moisture vapor permeable backing may not be required. As a result, some other potentially useful backing materials may include, e.g., metallocene polyolefins and SBS and SIS block copolymer (e.g., KRATON type) materials could be used.

Regardless, however, it may be preferred that the backings be kept relatively thin to, e.g., improve conformability. For example, it may be preferred that the backings be formed of (e.g., consist essentially of) polymeric films with a thickness of 200 micrometers or less, or 100 micrometers or less, potentially 50 micrometers or less, or even 25 micrometers or less.

### Pouch

In most embodiments, the pouch is a thermoplastic polymer. The pouch may be formed from materials similar to those described with respect to the backing. In one embodiment, the pouch is a self supporting substrate made of a thermoplastic polymer.

Thermoplastic materials suitable for use as the pouch include, for example, polyolefins (such as polyethylene); natural and synthetic rubbers (for example, styrene butadiene rubber or butyl rubber, polyisoprene, polyisobutylene, polybutadiene, polychloroprene, acrylonitrile/butadiene as well as functionalized elastomers such as carboxyl or hydroxyl modified rubbers, and the like); silicones including but not limited to polydimethylsiloxanes; styrenic block copolymers (for example, styrene-isoprene-styrene or styrene-ethylene/butylene-styrene block copolymer); polyurethanes including but not limited to those based on aliphatic isocyanate and aromatic isocyanate; and combinations thereof. Thermoset polymers may also be used.

In most embodiments, the pouch is translucent or transparent. In embodiments where the pouch is a self supporting substrate, the thermoplastic materials can be are semi-rigid with a percent elongation from 100% to 500% and a modulus from 10,000 to 400,000. Materials outside these parameters may not hold the shape of the self supporting substrate once formed.

In embodiments where the pouch is a self supporting substrate, the material used to form the self supporting substrate is generally substantially more rigid than the backing layer. In one embodiment, the thickness of the material is less than 6 mil, more preferably less than 5 mil. In most embodiments, the substrate material thickness is greater than 1 mil. In general, the pouch materials can include, but are not limited to, an elastic film, a non-elastic film, non-woven fibrous web, woven fibrous web, knits, and polyethylene/vinyl acetate copolymer-coated papers and polyester films.

The pouch is adhered or attached to the backing layers either directly or by an adhesive layer. The pouch can be pre-formed, such as by a mold process, then attached to the backing layers by an adhesive layer. Other ways of attaching the pouch include irreversible heat bonding or ultrasonically welding to the backing layer.

A pouch can be formed from a mold or other formed structure to form the desired construction. In one embodiment, a polyethylene film can be held between two plates with a designated open area. The polyethylene film is heated above its softening point but below its melting point. A plunger optionally with the desired corrugated shape can be pushed into the softened film to a depth that corresponds with the desired profile. After the polyethylene film cools below its softening point, the plunger is removed.

### Pressure Sensitive Adhesive

Suitable adhesive for use in wound dressing articles include any adhesive that provides acceptable adhesion to skin and is acceptable for use on skin (e.g., the adhesive should preferably be non-irritating and non-sensitizing). Suitable adhesives are pressure sensitive and in certain embodiments have a relatively high moisture vapor transmission rate to allow for moisture evaporation. Suitable pressure sensitive adhesives include those based on acrylates, polyurethanes, KRATON and other block copolymers, silicones, rubber based adhesives (including natural rubber, polyisoprene, polyisobutylene, butyl rubber etc.) as well as combinations of these adhesives. The adhesive component may contain tackifiers, plasticizers, rheology modifiers as well as active components including for example an antimicrobial agent.

The pressure sensitive adhesives that may be used in the wound dressings may include adhesives that are typically applied to the skin such as the acrylate copolymers described in U.S. Pat. No. RE 24,906, particularly a 97:3 isooctyl acrylate:acrylamide copolymer. Another example may include a 70:15:15 isooctyl acrylate: ethyleneoxide acrylate:acrylic acid terpolymer, as described in U.S. Pat. No. 4,737,410 (Example 31). Other potentially useful adhesives are described in U.S. Pat. Nos. 3,389,827; 4,112,213; 4,310,509; and 4,323,557. Inclusion of medicaments or antimicrobial agents in the adhesive is also contemplated, as described in U.S. Pat. Nos. 4,310,509 and 4,323,557.

Silicone adhesive can also be used. Generally, silicone adhesives can provide suitable adhesion to skin while gently removing from skin. Suitable silicone adhesives are disclosed in PCT Publications WO2010/056541 and WO2010/056543, the disclosure of which are herein incorporate by reference.

The pressure sensitive adhesives may, in some embodiments, transmit moisture vapor at a rate greater to or equal to that of human skin. While such a characteristic can be achieved through the selection of an appropriate adhesive, it is also contemplated that other methods of achieving a high relative rate of moisture vapor transmission may be used, such as pattern coating the adhesive on the backing, as described in U.S. Pat. No. 4,595,001. Other potentially suitable pressure sensitive adhesives may include blown-micro-fiber (BMF) adhesives such as, for example, those described in U.S. Pat. No. 6,994,904. The pressure sensitive adhesive used in the wound dressing may also include one or more areas in which the adhesive itself includes structures such as, e.g., the microreplicated structures described in U.S. Pat. No. 6,893,655.

Issued U.S. Pat. Nos. 3,645,835 and 4,595,001, the disclosures of which are hereby incorporated by reference, describe methods of making such films and methods for testing their permeability. Preferably, the film/adhesive composite should transmit moisture vapor at a rate equal to or greater than human skin. Preferably, the adhesive coated film transmits moisture vapor at a rate of at least 300 g/m²/24 hrs/37° C./100-10% RH, more preferably at least 700 g/m²/24 hrs/37° C./100-10% RH, and most preferably at least 2000 g/m²/24 hrs/37° C./100-10% RH using the inverted cup method as described in U.S. Pat. No. 4,595,001.

### Absorbent Materials

An absorbent material may also be used in conjunction with the medical dressings described herein. An absorbent material may be the same as the wound packing material (described below) or may be a separate element. The absorbent materials can be manufactured of any of a variety of materials including, but not limited to, woven or nonwoven cotton or rayon. Absorbent pad is useful for containing a number of substances, optionally including antimicrobial agents, drugs for transdermal drug delivery, chemical indicators to monitor hormones or other substances in a patient, etc.

The absorbent may include a hydrocolloid composition, including the hydrocolloid compositions described in U.S. Pat. Nos. 5,622,711 and 5,633,010, the disclosures of which are hereby incorporated by reference. The hydrocolloid absorbent may comprise, for example, a natural hydrocolloid, such as pectin, gelatin, or carboxymethylcellulose (CMC) (Aqualon Corp., Wilmington, Del.), a semi-synthetic hydrocolloid, such as cross-linked carboxymethylcellulose (X4ink CMC) (e.g. Ac-Di-Sol; FMC Corp., Philadelphia, Pa.), a synthetic hydrocolloid, such as cross-linked polyacrylic acid (PAA) (e.g., CARBOPOL™ No. 974P; B.F. Goodrich, Brecksville, Ohio), or a combination thereof. Absorbent materials may also be chosen from other synthetic and natural hydrophilic materials including polymer gels and foams.

### Carriers/Delivery Systems:

In some instances, the backings used in the medical dressings of the present invention may be so flexible and supple such that when a release liner is removed from the backing, the backing may tend to fold and adhere to itself, interfering with the smooth, aseptic application of the dressing to a patient's skin.

Various delivery systems have been proposed to address this problem such as those disclosed in U.S. Pat. No. 4,485,809; U.S. Pat. No. 4,600,001; and EPO Publication No. 0 051 935. Carrier-type delivery systems such as those described in U.S. Pat. No. 6,685,682 offer an alternative delivery system for use with conformable backings.

Alternative carriers and/or delivery systems may include frames, handles, stiffening strips, etc. as disclosed in issued U.S. Pat. Nos. 6,742,522; 5,979,450; 6,169,224; 5,088,483; 4,598,004; D 493,230; etc. Still another potentially suitable delivery system may be described in U.S. Patent Application Publication No. 2007/0156075 A1. In some instances, the backings can be delivered linerless as described in, e.g., U.S. Pat. No. 5,803,086.

The carrier material used to supply the carriers for dressings is preferably more rigid than the backing to prevent the backing from wrinkling during application. The carrier material can also be heat-sealable to the backing, with or without the low adhesion coating described below, for the purpose of manufacturing. In general, the carrier materials can include, but are not limited to, ethylene vinyl acetate copolymer or ethylene acrylic acid coated papers and polyester films.

The backing layer optionally also include a low adhesion coating on a top face of the backing, which is coated as a solution of polyvinyl N-octadecyl carbamate and a blend of silicone resins, as described in U.S. Pat. No. 5,803,086, which is incorporated by reference herein. It will also be understood that other coatings providing the low adhesion characteristics of the preferred coating could be substituted. The primary considerations in choosing any low adhesion coatings are their release characteristics and their compatibility with the attachment means between the carrier and the backing.

When the carrier is heat seal-bonded to the backing, the preferred low adhesion coating is compatible with the heat seal bond between the carrier and the backing and also retains its low adhesion characteristics after heat sealing. While it is preferred that the top face of the backing layer include a low adhesion coating, backing layers without such a coating with a carrier material attached thereto may be suitable.

### Release Liners

Release liners may be supplied with the medical dressings to protect the pressure sensitive adhesive used to attach the dressings to the patient and create the sealed cavity. Release liners that may be suitable for use in the medical dressing can be made of supercalendered kraft paper, glassine paper, polyethylene, polypropylene, polyester or composites of any of these materials.

The liners are preferably coated with release agents such as fluorochemicals or silicones. For example, U.S. Pat. No. 4,472,480 describes low surface energy perfluorochemical liners. The liners may preferably be in the form of papers, polyolefin films, polyolefin coated paper or polyester films coated with silicone release materials. Examples of commercially available silicone coated release liners are POLY SLIK™ silicone release on polyolefin coated papers, FL2000™ silicone release on film, and STICK-NOT™ silicone release on supercalendered kraft paper, all available from Loparex Inc., (Willowbrook, Ill.); silicone coated supercalendered kraft paper from Akrosil, (Menasha, Wis.); and silicone release film from Huhtamaki Florchheim, (Florchheim, Germany). Another potential liner is silicone coated (1630) low density polyethylene available from Huhtamaki.

The selection of a specific release liner may be made in conjunction with the selection of a pressure sensitive adhesive. Those skilled in the art will be familiar with the processes of testing a new adhesive against different liners or a new liner against different adhesives to arrive at the combination of qualities desired in a final product. U.S. Pat. No. 4,472,480 describes considerations pertinent to the selection of a perfluoropolyether release liner.

### Valves

The valves optionally provided in connection with the medical dressings have a relatively low profile or height. The low profile of the valves preferably reduces the likelihood that the medical dressing will be disturbed by external forces (from, e.g., bedding, clothing, etc.). The low profile valves may also improve patient comfort where, for example, the dressing is placed in a location on which the patient's weight rests while sitting, lying, and/or standing.

To retain a negative pressure within the sealed environments, it may be preferred that the openings in the medical dressings be one-way valves. In other words, it may be preferred that the valve allows fluid flow in one direction (out of the sealed environment) and restricts or prevents flow in the opposite direction (into the sealed environment). Alternatively, the valve allows fluid flow in one direction (into the sealed environment) and restricts or prevents flow in the opposite direction (out of the sealed environment).

Many valves can be used in the medical dressings described herein. For example, valves such as those known as “Goglio” type or “Raackmann” type valves may be used in connection with the present invention. Goglio-type valves are available, for example, from Bosch, Wipf, and Wico; Raackmann-type valves are available, for example, from Amcor. Other potentially suitable valves may include duckbill or umbrella valves (examples of which are those available from Vernay Laboratories, Inc., Yellow Springs, Ohio). Still other examples of suitable vacuum valves may include those described in U.S. Pat. Nos. 6,913,803; 6,733,803; 6,607,764; and 6,539,691, each of which is incorporated herein by reference in its entirety.

### Barrier Elements

Another optional element that may be included with the medical dressings of the present invention are barrier elements that may be placed proximate the valves of the medical dressings. The barrier elements may preferably function to filter materials from wound exudates that may otherwise cause the valves to become contaminated such that the valves do not re-close or seal after fluids are removed from the sealed environments defined by the medical dressings. The barrier elements may be provided attached to the medical dressings (e.g., within the cavity of the self supporting substrate) or they may be provided with the medical dressings in an unattached form such the barrier elements can be placed during delivery of the medical dressings to a patient. Examples of some materials that may be filtered by the barrier elements may include, e.g., clotted blood, loose tissue, wound packing, etc.

The barrier elements may be provided using a variety of different materials. Examples of some potentially suitable materials for the barrier elements may include, e.g., fabrics (e.g., gauze, nonwoven fabrics, woven fabrics, knitted fabrics, etc.), foams, etc. The barrier elements may also potentially incorporate absorbent materials such as, e.g., hydrogels, hydrocolloids, etc. In some embodiments, the barrier elements may be resiliently compressible, such that the barrier elements can also optionally function as ballast components to assist in maintaining a negative pressure in the sealed environment as described herein.

To accommodate an potentially adjustable pouch, the barrier could be cut-to-fit within the pouch or the barrier could itself have cuts to allow for expansion within the pouch.

### Septum Element

A septum element may be attached to the medical dressing backing to provide resealable access to the sealed environment through the medical dressing (e.g. a septum). The medical dressing can include only one or more than one septum element. The septum elements may, in some embodiments, be found on only one side of the backing or on both sides of the backing. In still other embodiments, one or more pairs of septum elements may be located on both the interior surface and the external surface of the backing, directly opposed from each other.

### Medical Dressing Kits

The medical dressings may potentially be supplied in the form of a kit with one or more of the optional components. The kit may preferably be provided in a sealed package (e.g., bag, pouch, tray, etc.). The kit includes one or more medical dressings as described herein.

The kit may also include one or more pumps that can be used in conjunction with the medical dressings. The kit may also include one or more traps that may be used with the one or more pumps to retain fluids (e.g., liquids) that may be removed from sealed environments defined by the dressings over wounds. In other embodiments, the kits may include one or more fluid traps, but no pumps where, for example, the user has a reusable pump that can be used with the trap or traps supplied in the kit with the dressings. The kits may also potentially include one or more fittings adapted for attachment to the external surfaces of the dressings as discussed herein, where the fittings can be used to provide connections between the valves in the dressings and the pumps.

### Active Agents

The medical dressing may optionally include at least one of a number of actives including for example, medicaments, anti-infective agents, antimicrobials, antiseptics (for example polyhexamethylene biguanide (hereinafter, “PHMB”), chlorhexidine, silver, iodine, an iodophor, benzalkonium chloride, hydrogen peroxide as well as the antiseptics disclosed in the following pending applications: US 2005/0089539, US2006/0051385, US2006/0052452, and US2006/0051384 which are incorporated herein by reference), antibiotics, analgesics, local anesthetics, anti-inflammatory agents, healing factors, vitamins, growth factors, enzyme inhibitors such as matrix metalloproteinase (MMP) inhibitors, and nutrients and/or one of a microbead packing and/or absorbent foam. Such actives may be introduced by elution off of any portion of the wound dressing including the backing, adhesive or barrier, or from a separate storage chamber that allows controlled introduction of the medication into the wound space due to the reduced pressure environment. Alternatively, medication may be introduced as taught in U.S. Pat. No. 6,867,342 or by injecting the medication directly through the dressing.

In some instances, it may be desirable to deliver one or more active agents to the sealed cavity formed by the pouch. The active agents may be provided as a fluid and/or may be carried within a fluid that is delivered to the internal volume. Some potentially suitable active agents may include, e.g., antimicrobials, antibiotics, analgesics, healing factors such as vitamins, growth factors, nutrients and the like. Examples of other potentially suitable agents may be described in U.S. Pat. No. 6,867,342.

If delivered, an active agent (or agents) could be supplied to the sealed cavity continuously or intermittently. For example, an active agent could be delivered to the sealed cavity and allowed to remain in place (i.e., resident) for a selected period of time (e.g., several hours) followed by, e.g., delivery of a second active agent, delivery of negative pressure therapy, etc. The initial active agent could be removed before delivery of the second agent or it could be allowed to remain in place. Alternatively, the sealed cavity could be rinsed with, e.g., saline or another flushing solution before placing the sealed environment in a negative pressure condition, before delivery of a second agent, etc.

### Wound Pumps

As discussed herein, the medical dressings may be used for negative pressure wound therapy by providing an opening or port, such as a valve, in the medical dressing through which fluid can be removed from a sealed environment defined by the medical dressing. The fluid can be removed from the sealed environment using a pump that can be periodically attached to the medical dressing. It may be preferred that the pump include a seat that can seal against the external surface of the backing of the medical dressing to provide a fluid-tight seal.

The pumps used in connection with the medical dressings may take any suitable form. In some embodiments, the pumps may be portable, self-contained devices, while in other embodiments the pumps may be fixed, stationary systems. In some instances, fluids may even be removed from a sealed environment defined by the medical dressings using suction developed by a person using their mouth (in, e.g., a battlefield or other remote location). In one embodiment the pump is a pump as disclosed in Applicant's copending patent application WO2009/124100.

Examples of some potentially suitable pumps that may be used with and/or supplied in a kit with the medical dressings may include the pumps described in U.S. Patent Application Publication No. U.S. 2007/0209326 (Tretina). Although the pumps described in the document identified above include a power source (e.g., a battery), pumps may be manually powered. Examples of some other potentially suitable manually powered pumps may include, e.g., devices that include resilient cavities that can be compressed and, when returning to their pre-compression states, provide a vacuum force at the inlet of the pump (e.g., bulbs, hemovacs, etc.).

In some embodiments, the pumps may preferably include one or more traps or fluid collection components capable of collecting and retaining liquids (and, in some embodiments, gases) removed from the sealed environments defined by the medical dressings. The traps may be integral with the pumps in some embodiments, while in other embodiments the traps may be separate from the pumps such that the traps may be replaced without requiring replacement of both the pumps and the traps. Examples of some potentially suitable traps that are designed to separate liquids from the removed fluids may be described in, e.g., U.S. Patent Application Publication Nos. U.S. 2007/0209326 (Tretina) and U.S. 2007/0172157 (Buchman).

It may be preferred that the medical dressings and any pumps used therewith to remove fluids from sealed environments be capable of quickly connecting with each other to form a fluid-tight seal during removal of fluids from the sealed environments defined by the medical dressings. The medical dressings and pumps may include more conventional connections/fittings to provide a fluid-tight connection between the pumps and the medical dressings. Such fittings may be useful where, e.g., the pump is to be connected to the medical dressing for an extended period of time, e.g., for more than 2 minutes. In such an embodiment, the medical dressing kit may include a fitting that attaches to the external surface of the backing using, e.g., a pressure sensitive adhesive, etc. The fitting may, for example, include a tubing connector, Luer lock fitting, etc. designed for longer-term connection to a pump. The adhesive used to attach the fitting to the medical dressing may be releasable, i.e., the fitting may potentially be removed from the dressing while the dressing remains in place over a wound, such that any sealed environment defined by the medical dressing remains intact during removal of the fitting.

In some embodiments, the pump is a low cost disposable pump. Disposing of the pump with each dressing change reduces the risk of bacterial contamination of the wound and transmission to other patients.

A check valve or other means may be required to regulate pressure, particularly for pumps able to create a vacuum of more than 100 mmHg below atmospheric pressure. This may be accomplished via a check valve that opens at a predetermined pressure drop and allows air into the wound bed. If a check valve is used it preferably has a membrane element that will filter out microorganisms and prevent them from entering the wound bed.

Alternatively the pump is equipped with a pressure sensor and a control circuit that slows the pump speed at a predetermine pressure set point. The set point is preferably variable and easily set by the clinician. A read out of the pressure may be desired. Alternatively, the pump is self limiting and unable to create a vacuum more than the desired maximum vacuum, e.g. more than about 150 mmHg.

The pump is preferably programmable to pull a continuous, intermittent or variable vacuum. For example, the pump could be programmed to pull and hold a vacuum of 100 mmHG or be programmed to pull a vacuum of 150 mmHg for a period of time following by a period of time at a vacuum of 25 mmHg below atmospheric pressure in an oscillatory fashion.

In one embodiment, the pump is secured directly to the wound dressing either through the interior portion of the dressing or at the periphery. In either case an inlet tube may be unnecessary. The pump also can be remote from the dressing and attached via an inlet tube. In such case, the pump may have multiple inlets and exit ports and/or multiple pumps may be employed on a single dressing. Such inlet means may be a simple tube which passes fluid from the wound bed into the pump. The inlet of the inlet tube may then need to be protected by a porous filter element. The inlet means may be a simple flexible tube or may be other means such as the fluid control articles described in U.S. Pat. No. 6,420,622 or the drain tubes described in U.S. Pat. No. 6,420,622.

The pump exit may in fluid communication with a reservoir designed to collect the excess wound fluid. The fluid reservoir may be a vented rigid container, a flexible container, or a vented flexible container. Preferred fluid reservoirs can be a flexible reservior similar to those used in ostomy appliances such as those disclosed in U.S. Pat. No. 7,214,217. The pouches may even be flushable as disclosed in U.S. Pat. No. 7,179,245. However, the fluid reservoir may be as simple as a vacuum canister such as used routinely in surgery, a canister such as described in U.S. Pat. No. 4,569,674. The collection pouch can be constructed of any suitable polymeric material but is preferably an odor barrier such as disclosed in U.S. Pat. No. 7,270,860. Furthermore, the collection reservoir may have a means for alerting the patient or caregiver that it should be changed. This alert can be an electronic means or a passive means.

A sample port may be provided between the pump and the fluid reservoir or on the reservoir itself for easily obtaining a sample for analysis. For example, a “T” shaped tubing may be provided in the exit line or a simple valved port on the fluid reservoir with a lure lock for attaching a syringe may be used. The sample can be used for analysis of chemical or physical properties of the wound fluid in order to assess healing or for further treatment means.

### Wound Packaging Material

In some embodiments, the medical dressings may be provided with wound packing material in place of a barrier element or in addition to a barrier element. The wound packing material may, in some embodiments, also function as a barrier element as described herein (although this function is not required). In some embodiments, the wound packing material may be resiliently compressible, such that the wound packing material can also optionally function as a ballast component to assist in maintaining a negative pressure in the sealed environment as described herein. For example, when a vacuum is applied the resilient packing will be compressed. When the vacuum is removed and the valve closed to seal the wound cavity the resilient packing will still provide an expansion force in order to return to its non-compressed state. This expansion will serve to create or help maintain a vacuum for a period of time.

Wound packing materials may be useful where, e.g., the wound to be contained within the sealed environment is a chronic wound that is in the form of a significant depression (which may, in some instances be tunneled under the surrounding skin). When treating such wounds, it may be desirable to provide wound packing material in the wound before applying a medical dressing to create a sealed environment over the wound.

The wound packing material may preferably be flexible such that it can fill and/or conform to the shape of the wound. The wound packing may be absorbent or non-absorbent. The wound packing may be capable of providing passageways through which fluids can pass. Some potentially suitable examples of wound packing materials may include fully or partially reticulated foam (e.g., open cell polyurethane foams, etc.), fabric (e.g., gauze, mesh, woven, knit, or nonwoven materials), particulate materials, beads, etc. that may be placed in a wound to fill the internal volume. If provided in particulate or bead form, the particulates or beads may, in some embodiments, be contained within a flexible bag or other structure to facilitate removal of the wound packing (unless, e.g., the wound packing material is bioabsorbable and/or biodegradable). A preferred polyurethane foam may be hydrophilic and capable of spontaneously absorbing deionized water such as WILSORB foam (available from Illbruck). Preferred hydrophilic packing components will absorb a 100 microliter drop of deionized water when gently placed in contact with the foam in less than 60 seconds and preferably in less than 30 seconds.

Polyvinylalcohol (PVA) open cell foams may also be used. A preferred fabric is nonwoven fabric and more preferably a lofted nonwoven fabric having resiliency such that when compressed to 50% of its thickness rebounds to 90% or greater of the original thickness in less than 10 seconds and preferably in less than 1 second. A preferred lofted resilient nonwoven has physical properties similar to 3M Buff Puff™ Facial Sponge. These structures may be treated to be hydrophilic and spontaneously wet with water. In some preferred embodiments the intermediate material may include several hydrophilic colloid materials to absorb fluids. In other embodiments the intermediate layers are preferably hydrophobic in order to retard tissue ingrowth. One skilled in the art will appreciate that there may be a number of materials suitable for the intermediate layer to achieve various objectives including combinations of the materials mentioned above and combinations that include other materials.

The wound packing can be secured directly to the dressing. For example, the wound packing can be secured via the adhesive layer. In this embodiment the wound packing is placed at least over the portion of the dressing, and may be located where the pump inlet conduit will be located. This may be in the interior of the dressing or may be located at the periphery.

If the barrier element and/or wound packing materials are provided in a form such that they are not attached to the medical dressing, the medical dressing may be provided in the form of a kit including the medical dressing and the separate barrier element and/or wound packing. In using such a kit, the barrier element and/or wound packing may be attached to the medical dressing before the medical dressing is delivered to a patient. Alternatively, the barrier element and/or wound packing may be placed on or in the wound, with the medical dressing deployed over the wound after the barrier element and/or wound packing is/are in position.

Although specific embodiments of this invention have been shown and described herein, it is understood that these embodiments are merely illustrative of the many possible specific arrangements that can be devised in application of the principles of the invention. Numerous and varied other arrangements can be devised in accordance with these principles by those of ordinary skill in the art without departing from the spirit and scope of the invention. Thus, the scope of the present invention should not be limited to the structures described in this application, but only by the structures described by the language of the claims and the equivalents of those structures.

1. (canceled)

2. A medical dressing comprising:
a first backing comprising a conformable film formed in a generally C-shape having an first open end and a first closed end;
a second backing comprising a conformable film formed in a generally C-shape having a second open end and a second closed end;
wherein in use the first open end of the first backing is laterally aligned with the second open end of the second backing so as to create an opening surrounded by at least a portion of the first backing and second backing.

3. The medical dressing of claim 2, further comprising an adhesive layer on at least a portion of a first major surface of the first backing and on at least a portion of a first major surface of the second backing.

4. The medical dressing of claim 3, wherein the adhesive is at a perimeter of the medical dressing.

5. The medical dressing of claim 2, further comprising a pouch secured to a second major surface of the first backing and a second major surface of the second backing and completely surrounding the opening, wherein the pouch forms a cavity at the opening.

6. The medical dressing of claim 5, wherein the pouch is a self-supporting substrate.

7. The medical dressing of claim 5, further comprising a barrier element for placement within the pouch.

8. The medical dressing of claim 7, wherein the barrier element is secured within the pouch.

9. The medical dressing of claim 7, wherein the barrier element is laterally expandable or collapsible.

10. The medical dressing of claim 9, wherein the barrier element includes off-set cuts to allow for lateral expansion.

11. The medical dressing of claim 7, wherein the barrier element is an absorbent material.

12. The medical dressing of claim 5, wherein the pouch is laterally expandable or collapsible.

13. The medical dressing of claim 12, wherein the pouch includes a corrugated portion comprising alternating ridges and valleys to allow the pouch to expand or contract to accommodate various sizes of openings.

14. The medical dressing of claim 5, wherein the pouch includes a first portion secured with the first backing and a second portion secured with the second backing, wherein in use the first portion of the pouch and second portion of the pouch are in overlapping relation to one another to form the cavity.

15. The medical dressing of claim 2, further comprising a third thin film backing, wherein in use the first backing, second backing, and third backing are in overlapping relation to one another and entirely surround the opening to form the window.

16. The medical dressing of claim 2, further comprising a carrier releasably attached to at least a portion of the first backing and second backing.

17. The medical dressing of claim 5, wherein the pouch further comprises a valve allowing for fluid flow.
